(19) United States
(12) Patent Application Publication
(10) Pub. No.: US 2024/0013679 A1
(43) Pub. Date: Jan. 11, 2024

(54) SMART SENSOR-EQUIPPED PHANTOM FOR PERFORMANCE ASSESSMENT OF MEDICAL PROCEDURES

(71) Applicant: THE JOHNS HOPKINS UNIVERSITY, Baltimore, MD (US)

(72) Inventors: Ehsan AZIMI, Baltimore, MD (US); Peter KAZANZIDES, Lutherville, MD (US); Zhiyuan NIU, Baltimore, MD (US)

(73) Assignee: THE JOHNS HOPKINS UNIVERSITY, Baltimore, MD (US)

(21) Appl. No.: 18/247,579

(22) PCT Filed: Sep. 28, 2021
(86) PCT No.: PCT/US2021/052398

§ 371 Date: Mar. 31, 2023

Related U.S. Application Data
(60) Provisional application No. 63/086,719, filed on Oct. 2, 2020.

(51) Int. Cl. G09B 23/28; A61B 34/20

(52) U.S. Cl. CPC G09B 23/285; A61B 34/20; A61B 2034/2055; A61B 2034/2065

(57) ABSTRACT

A system includes: a phantom object; one or more sensors within the phantom object; and a processor, a computer readable memory, a non-transitory computer readable storage medium associated with a computing device in communication with the phantom object, and program instructions executable by the computing device to cause the computing device to perform operations including: detecting a medical instrument within the phantom object based on sensor data captured by the one or more sensors; measuring a distance between the medical instrument and a target point based on the sensor data; and storing or outputting information identifying the distance between the medical instrument and the target point.

### CROSS-REFERENCE TO RELATED APPLICATIONS

This application claims priority to U.S. Provisional Patent Application No. 63/086,719 filed on Oct. 2, 2020, which is hereby incorporated herein in its entirety.

### BACKGROUND

In various surgical procedures, devices, such as catheters, may need to be precisely inserted into the patient. Surgeons typically may rely on their general knowledge of anatomy and relatively crude and uncertain measurements for locating internal anatomical targets. One example is for a ventriculostomy surgical procedure, in which the surgeon must insert a catheter into the ventricle to drain cerebrospinal fluid (CSF). In this procedure, surgeons make measurements relative to cranial features to determine where to drill into the skull and then attempt to insert a catheter as perpendicular to the skull as possible.

Although ventriculostomy is one of the most commonly performed neurosurgical procedures, studies have shown a large number of misplaced catheters and many cases in which multiple attempts (passes) were required to hit the target (e.g., ventricle). Misplacement of the catheter can cause hemorrhage, infection and other injuries to the patient. These risks may be higher when the procedure is performed by a less experienced surgeon, which may occur in emergency situations. Training surgeons to perform such medical procedures may include supervised training on live patients, which may be risky.

### SUMMARY

In one example aspect, a computer-implemented method includes: detecting a medical instrument within a phantom object based on sensor data captured by one or more sensors implemented within the phantom object; measuring a distance between the medical instrument and a target point based on the sensor data; and storing or outputting information identifying the distance between the medical instrument and the target point.

In another example aspect, a computer program product includes a computer readable storage medium having program instructions embodied therewith. The program instructions are executable by a computing device to cause the computing device to perform operations including: detecting a medical instrument within a phantom object based on sensor data captured by one or more sensors implemented within the phantom object; measuring a distance between the medical instrument and a target point based on the sensor data; and storing or outputting information identifying the distance between the medical instrument and the target point.

In another example aspect, a system includes: a phantom object; one or more sensors within the phantom object; and a processor, a computer readable memory, a non-transitory computer readable storage medium associated with a computing device in communication with the phantom object, and program instructions executable by the computing device to cause the computing device to perform operations including: detecting a medical instrument within the phantom object based on sensor data captured by the one or more sensors; measuring a distance between the medical instrument and a target point based on the sensor data; and storing or outputting information identifying the distance between the medical instrument and the target point.

### BRIEF DESCRIPTION OF THE DRAWINGS

FIGS. 1A-1E illustrate an overview of an example implementation of a phantom object used in conjunction with aspects of the present disclosure.

FIG. 2 illustrates an example environment as described herein.

FIG. 3 illustrates an example flowchart of a process for measuring distance between a medical instrument and an anatomical target within a phantom object.

FIG. 4 illustrates an example virtual navigation system which may be evaluated using the techniques described herein in accordance with aspects of the present disclosure.

FIG. 5 shows coronal views of two synthetic CT scans (left: nominal; right: abnormal).

FIG. 6 illustrates example components of a device that may be used within the environment of FIG. 2.

### DETAILED DESCRIPTION

In various surgical procedures, medical instruments or tools, such as catheters, may need to be precisely inserted into the patient to minimize the risk of medical complications (e.g., infections, hemorrhages, etc.). More specifically, a medical instrument may need to be placed at a precise target location. The placement of these devices may be inaccurate, especially when the surgical procedure is being performed by a less experienced surgeon or doctor. Also, training surgeons to perform such medical procedures may include supervised training on live patients, which may be risky. Accordingly, the systems and/or methods, described herein, may measure the accuracy of medical instrument/tool placement using a sensor-equipped phantom object. More specifically, the systems and/or methods may include a sensor-equipped phantom object (e.g., a physical replica of an organ) that may be used to assist in locating internal anatomical targets (e.g., as part of training for performing medical procedures). Additionally, or alternatively, the phantom object, in accordance with aspects of the present disclosure, may be used to measure the performance of navigation systems or other surgical techniques used to locate internal anatomical targets (e.g., a virtual navigation system, an anatomical virtual navigation system, a mechanical guide, etc.). That is, aspects of the present disclosure may be used to aid in training and/or for system validation procedures.

In some embodiments, the phantom object may include one or more sensors (e.g., cameras, object detection sensors, light sensors, infrared sensors, etc.) within the phantom object whereby the interior of the replica organ may be displayed to assist a surgeon in locating internal anatomical targets (e.g., as part of a training exercise). In some embodiments, the systems and/or methods, described herein, may measure the distance between a medical instrument insertion point within the phantom object, and a target location (e.g., a location of an anatomical target) within the phantom object. As one, illustrative, non-limiting example, the phantom object may be a replica of a skull in which a target location may be a ventricle. As described herein, the phantom object may include one or more cameras to view the inside of phantom object (e.g., the inside of a replica skull). In some embodiments, the phantom object may further include markers (e.g., posts or protrusions) that may represent an anatomical target. In this example, the phantom object may be used for a neurosurgery training process to train a surgeon on catheter insertion for a ventriculostomy surgical procedure and to provide feedback identifying the accuracy of the placement of the catheter in relation to a target (e.g., a ventricle). In other embodiments, different sensors may be used in place of or in addition to the camera or cameras.

Aspects of the present disclosure may include an application that communicates with the phantom object and receives video data from the cameras within the phantom object. In some embodiments, the application may present a user interface whereby a user may view the interior of the phantom object, and define a point or area corresponding to an anatomical target. Additionally, or alternatively, the anatomical target may be automatically detected based on the markers within the phantom object. For example, in some embodiments, the application may incorporate image classification and/or recognition techniques to recognize one or more markers as an anatomical target. Also, during a procedure (e.g., a training or navigation system validation procedure), the application may detect a medical instrument (e.g., a catheter, needle, replica of a catheter or needle, and/or other type of medical instrument or tool) inserted into the phantom object. In some embodiments, the application may measure a distance from the medical instrument to the anatomical target. In this way, feedback may be provided that informs the user as to the accuracy of the insertion of the medical instrument.

As an example use case, in a training exercise, a user (e.g., medical student, resident, trainee, etc.) may insert the medical instrument into the phantom object to simulate performing a procedure (e.g., a ventriculostomy surgical procedure). During the training exercise, the application may display a level of accuracy of the insertion of the medical instrument to aid the user and to assist in improving technique and accuracy for future procedures (e.g., future training and/or real-life procedures). In another example use case, the application may indicate a level of accuracy of the insertion of the medical instrument to validate the performance of a medical device, such as a virtual navigation system. For example, the virtual navigation system may render a virtual catheter insertion path within a virtual headset, (e.g., an augmented reality (AR), virtual, mixed, and/or extended reality headset). The systems and/or methods, described herein, may measure the accuracy and performance of this virtual navigation system, (e.g., by measuring the distance between the virtual insertion path and the actual anatomical target) when a medical instrument is inserted along the virtual insertion path. The systems and/or methods, described herein, may provide feedback regarding the accuracy, and if the virtual navigation system is inaccurate, adjustments may be made to the virtual navigation system to improve the accuracy of the virtual rending of the insertion path. Additionally, or alternatively, the performance of other types of medical devices and/or navigation and assistance systems may be evaluated using the techniques described herein, such as tablet/smartphone based or projection-based systems, robotic systems for insertion of a medical instrument, etc.

In some embodiments, the systems and/or methods, described herein, may record video and/or capture photographs of a procedure in which a medical instrument is inserted into the phantom object. In this way, users may review their training procedure for improving their technique and accuracy. In some embodiments, the systems and/or methods may provide real-time feedback indicating the user's accuracy with respect to medical instrument insertion relative to a target location. Also, the user may view the inside of the phantom object during a procedure in real time to aid in training. As the user becomes more adept, the user may choose not to view the procedure in real time to more closely simulate a real-life procedure in which the user may not be able to view the inside of a live patient.

Certain embodiments of the disclosure will hereafter be described with reference to the accompanying drawings, wherein like reference numerals denote like elements. It should be understood, however, that the accompanying drawings illustrate only the various implementations described herein and are not meant to limit the scope of various technologies described herein. The drawings show and describe various embodiments of the current disclosure.

Embodiments of the disclosure may include a system, a method, and/or a computer program product at any possible technical detail level of integration. The computer program product may include a computer readable storage medium (or media) having computer readable program instructions thereon for causing a processor to carry out aspects of the present disclosure.

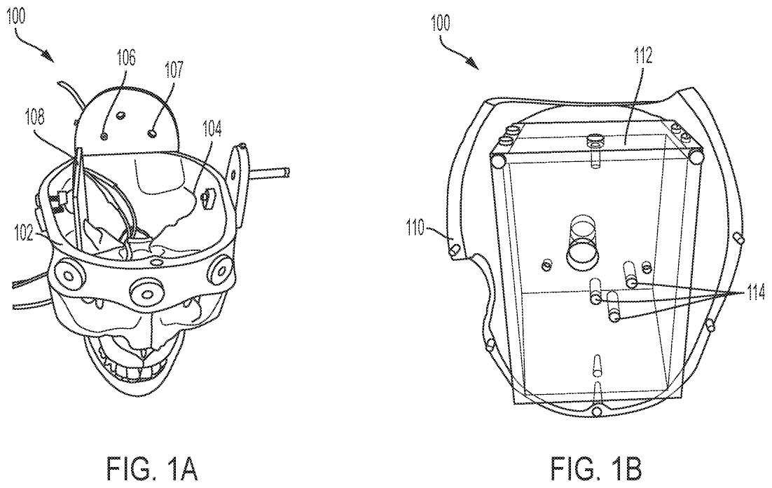

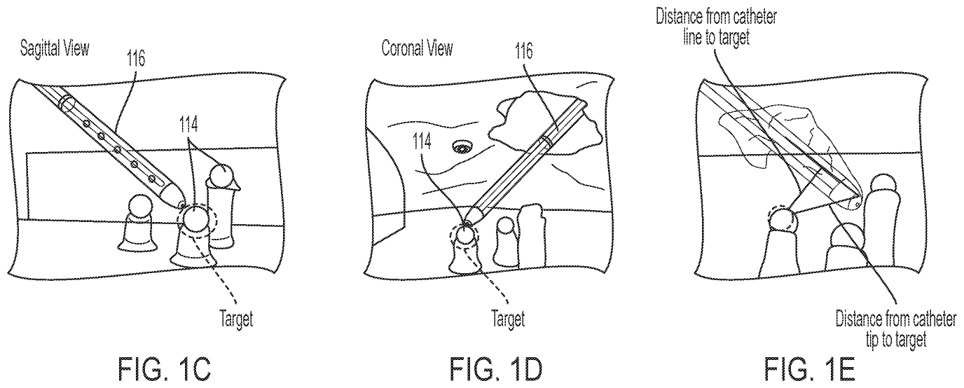

FIGS. 1A-1E illustrate an overview of an example implementation of a phantom object used in conjunction with aspects of the present disclosure. As shown in FIG. 1A, a phantom object 100 may be a replica of an organ. In the illustrative example shown in FIG. 1A, the phantom object 100 may be replica of a skull in which the phantom object 100 may be used to simulate a procedure involving the skull (e.g., ventriculostomy surgical procedure).

As further shown in FIG. 1A, the phantom object 100 may include a first exterior 102, a void 104, a sensor 106 located within a recess 107, and a light array 108. As shown in FIG. 1B, the phantom object 100 may include a second exterior 110 which may be a top portion of a skull replica. Within the second exterior 110, the phantom object 100 may include a frame 112, and markers 114. The second exterior 110 may be assembled over the first exterior 102. When assembled, the sensors 106 may be positioned such that interior of the phantom object 100 is viewable through the sensors 106. As described herein, the markers 114 may be posts with a spherical tip. The markers 114 may be removable or integrated within the phantom object 100 and may be used to calibrate a measuring system. Additionally, or alternatively, the markers 114 may be used to identify a target location or area within the phantom object 100.

In some embodiments, the phantom object 100 may be constructed from a material that simulates the properties of the corresponding organ (e.g., a skull). Additionally, or alternatively, the phantom object 100 may be constructed from any variety of suitable materials, plastics, polymers, etc. In some embodiments, the phantom object 100 may include additional or fewer components than those shown in FIGS. 1A and 1B. For example, the phantom object 100 may include a burr hole from which a medical instrument (e.g., catheter, needle, replica of a catheter or needle, etc.) may be inserted. Additionally, or alternatively, the phantom object 100 may not include a burr hole, so as to more closely similar a real-life scenario in which the burr hole may need to be formed.

In some embodiments, the phantom 100 can be constructed from a plastic skull with a cranial cap that is magnetically attached, as shown in FIG. 1A-1B. A clear acrylic box 112 can be inserted to hold gel that mimics the brain tissue. Three spheres 114 can be placed near the bottom of the acrylic box to use as targets.

FIGS. 1C-1E illustrate video and/or images captured by the sensors 106 (e.g., cameras) implemented within the phantom object 100. In some embodiments, the video and/or images may show different views from different sensors 106 positioned in various locations within the phantom object 100 when the second exterior 110 is attached to the first exterior 102. For example, FIG. 1C may illustrate a sagittal view and FIG. 1D may illustrate a coronal view. In some embodiments, the interface may graphically indicate a target location (e.g., as one of the markers within the phantom object 100). In some embodiments, the light array 108 may be provided to improve and support sensing and/or video/image capturing by the sensors 106.

In some embodiments, a computer vision system can be used to measure the 3D coordinates of the catheter and target, as well as to record videos, as shown in FIG. 1A-1B. One camera can be fixed on the left side of the skull, and the other can be on the back. Due to the different refractive indices between the gel and air, the intrinsic calibration of each camera can be separately performed with a checkerboard in the gel. The extrinsic parameters, which do not change with the medium, can be calibrated in air. The accuracy of the optical measurement system can be obtained by identifying each of the three spherical targets inside the skull and computing the distances between them. These three distances can be compared to the same distances computed in the CT scans to measure the accuracy.

FIG. 1C-1E shows representative images from the measurement software for a catheter insertion. During the experimental procedure, once the catheter reaches the guided position, two images can be captured. The target, catheter tip and a second point on the catheter (to determine its orientation) can be identified in both images. Consequently, the distances between the spherical target and the catheter tip, as well as the catheter line, can be obtained to evaluate the accuracy of the guidance system.

As described herein, the target location may be selected and defined by the user from within the interface via user inputs as part of a calibration process. In some embodiments, the target location may correspond to a location of one or more of the markers or may be completely independent and separate from the markers. Alternatively, the target location may be automatically detected based on one or more of the markers and software that automatically detects the one or more markers (e.g., based on object detection and/or any suitable image classification technique).

As further shown in FIGS. 1C-1E, a distance from a medical instrument 116 (e.g., a catheter or catheter replica) to a target may be presented. In the example of FIGS. 1C and 1D, the distance may be zero, as the medical instrument 116 is placed on the target. In some embodiments, the distance from different parts of the medical instrument 116 to the target may be determined and displayed. For example, referring to FIG. 1E, a distance from the catheter line to the target and/or the distance from the catheter tip to the target may be displayed. In the example of FIG. 1E, the target is defined as a different marker than the target marker defining the target in FIGS. 1C and 1D.

In some embodiments, the distance may be presented as a numerical value in any suitable units, and may be overlaid as text within the video image. As described herein, the medical instrument may be recognized by the application using any variety of suitable techniques (e.g., image/object recognition techniques, object tracking techniques, etc.). In some embodiments, the distance may be determined using a calibration technique in which the distances between the markers are known, and thus, the distance of each pixel in the image may be determined. In this way, the distance between the medical instrument and the target may be determined based on the number of pixels separating the medical instrument and the target in the images. Additionally, or alternatively, in some embodiments, three-dimensional coordinates of the medical instrument and the target may be determined using a stereo triangulation technique in which the configuration of the sensors is known, and thus, the distance between the medical instrument and the target may be determined. In some embodiments, information identifying the distance between the medical instrument and the target may be recorded and reported (e.g., in real time or at a later time for evaluation).

As described herein, the systems and/or methods, described herein may continuously measure the distance between the medical instrument 116 and the target as the medical instrument 116 is inserted into the phantom object 100. In some embodiments, the measured distance may be continuously reported on an image or video of the interior of the phantom object 100. In this way, during a procedure (e.g., a training procedure), real-time feedback may be provided to assist in the training. Also, any variety of audio and/or visual effects may be added to represent the distance between the medical instrument 116 and the target (e.g., a green color may be rendered to represent that the target has been “hit” by the medical instrument 116). In some embodiments, video and/or images captured by the sensors 106 may be recorded/saved. Also, any variety of reports may be generated that identify the distance between the medical instrument 116 and the target at various points in time during a procedure.

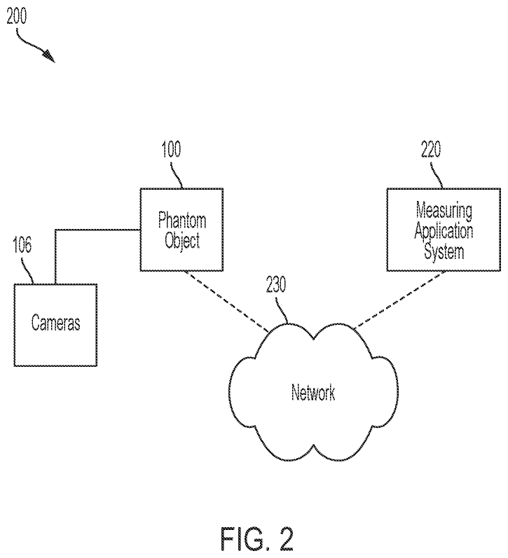

FIG. 2 illustrates an example environment in accordance with aspects of the present disclosure. As shown in FIG. 2, environment 200 includes a phantom object 100, a measuring application system 220, and a network 230.

The phantom object 100 may include a physical replica (e.g., of an organ) that is fitted with one or more sensors 106 (e.g., within the interior of the phantom object 100). As one illustrative example, the phantom object 100 may be a replica of a skull (e.g., as shown in the example of FIGS. 1A-1E). However, the phantom object 100 may be a replica of any organ or object. The sensors 106 may be cameras or other types of sensors from which image and/or video may be derived or from which other information may be obtained relevant to a procedure (e.g., lighting data, moisture data, object proximity data, force data, etc.). In some embodiments, the phantom object 100 may include communication devices to communicate via the network 230 (e.g., to transmit video/image data and/or other sensor data captured by the sensors 106).

The measuring application system 220 may include one or more computer devices that hosts an application for measuring the accuracy of medical instrument placement in relation to a target defined on or within the phantom object 100. As described herein, the measuring application system 220 may receive video/image data captured by the sensors 106 within the phantom object 100 and present the video/image data within a calibration interface in which one or more target points or areas may be defined within the phantom object 100 (e.g., with the assistance of markers within the phantom object 100, or independently of the markers). Once calibrated (e.g., when one or more target points or areas have been defined using the calibration interface), in operation, the measuring application system 220 may implement object detection techniques (e.g., pixel-based classification and/or other classification techniques) to detect a medical object viewed by the sensors 106 and measure a distance between the medical object and the one or more target points. The measuring application system 220 may store and/or output information identifying the measured distance such that the measured distance may be used for real-time feedback and/or post-operation feedback (e.g., to assist in training and/or evaluation of a medical device, such as a virtual navigation system).

The network 230 may include network nodes and one or more wired and/or wireless networks. For example, the network 230 may include a cellular network (e.g., a second generation (2G) network, a third generation (3G) network, a fourth generation (4G) network, a fifth generation (5G) network, a long-term evolution (LTE) network, a global system for mobile (GSM) network, a code division multiple access (CDMA) network, an evolution-data optimized (EVDO) network, or the like), a public land mobile network (PLMN), and/or another network. Additionally, or alternatively, the network 230 may include a local area network (LAN), a wide area network (WAN), a metropolitan network (MAN), the Public Switched Telephone Network (PSTN), an ad hoc network, a managed Internet Protocol (IP) network, a virtual private network (VPN), an intranet, the Internet, a fiber optic-based network, and/or a combination of these or other types of networks. In embodiments, the network 230 may include copper transmission cables, optical transmission fibers, wireless transmission, routers, firewalls, switches, gateway computers and/or edge servers.

The quantity of devices and/or networks in the environment 200 is not limited to what is shown in FIG. 2. In practice, the environment 200 may include additional devices and/or networks; fewer devices and/or networks; different devices and/or networks; or differently arranged devices and/or networks than illustrated in FIG. 2. Also, in some implementations, one or more of the devices of the environment 200 may perform one or more functions described as being performed by another one or more of the devices of the environment 200. Devices of the environment 200 may interconnect via wired connections, wireless connections, or a combination of wired and wireless connections.

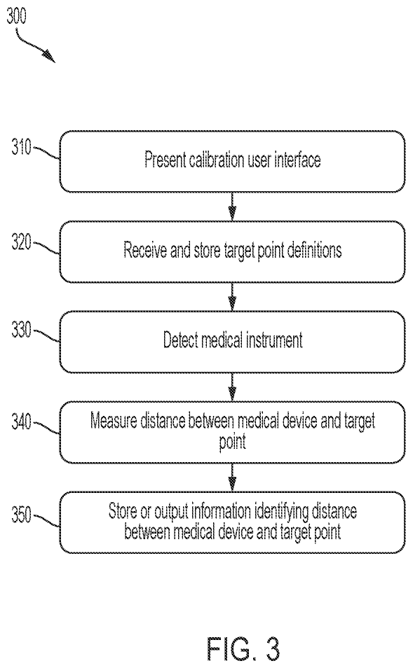

FIG. 3 illustrates an example flowchart of a process for measuring distance between a medical instrument and an anatomical target within a phantom object. The blocks of FIG. 3 may be implemented in the environment of FIG. 2, for example, and are described using reference numbers of elements depicted in FIG. 2. As noted herein, the flowchart illustrates the architecture, functionality, and operation of possible implementations of systems, methods, and computer program products according to various embodiments of the present disclosure.

As shown in FIG. 3, the process 300 may include presenting a calibration user interface (block 310). For example, the measuring application system 220 may present a user interface that displays a video feed of the inside of the phantom object 100.

The process 300 also may include receiving and storing target point definitions (block 320). For example, the measuring application system 220 may receive and store target point definitions. In some embodiments, within the interface presented at block 310, the user may provide user input to define one or more target points (e.g., anatomical targets). In some embodiments, the target points may be based on the location of the markers 114 within the phantom object 100 or may be a virtual space defined relative to, or independently of, the markers 114. Additionally, or alternatively, the measuring application system 220 may automatically detect the markers 114 (e.g., using image/object detection techniques, pixel-based classification techniques, etc.). In some embodiments, the measuring application system 220 may also receive information identifying the distance between the markers 114.

The process 300 further may include detecting a medical instrument (block 330). For example, the measuring application system 220 may detect a medical instrument (e.g., the medical instrument 116) within the field of view of the sensors 106 within the phantom object 100. In some embodiments, the medical instrument 116 may be placed within the field of view of the sensors 106 during a procedure (e.g., a training and/or validation procedure). For example, a user may insert the medical instrument 116 through an exterior of the phantom object 100 such that the medical instrument 116 is visible by the sensors 106 within the phantom object 100 (e.g., as shown in FIGS. 1C-1E). The measuring application system 220 may detect the medical instrument 116 using an object recognition technique (e.g., based on pixel-based classification), and/or using any other variety of techniques. The process 300 also may include measuring a distance between the medical instrument and target point (block 340). For example, the measuring application system 220 may measure a distance between the medical instrument and the target point. In some embodiments, the measuring application system 220 may determine a number of pixels in a video or image between the medical instrument and the target point. As one example, the measuring application system 220 may measure the distance based on the number of pixels separating the medical instrument and the target point and may multiply the number of pixels by a distance represented by each pixel. Additionally, or alternatively, the measuring application system 220 may measure three-dimensional coordinates of the medical instrument and the target point based on a stereo triangulation technique and determine the distance between the medical instrument and the target point.

The process 300 further may include storing or outputting the information identifying the distance between the medical instrument and the target point (block 350). For example, the measuring application system 220 may store or output the information identifying the distance between the medical instrument and the target point. In some embodiments, the information may be outputted during a real-time procedure and displayed within an interface that displays the video from the sensors 106 within the phantom object 100. In some embodiments, the information identifying the distance between the medical instrument and the target point may be overlaid on the video. The video and/or images of a procedure may also be saved for future reference.

In some embodiments, the measuring application system 220 may continuously perform steps 330-350 during a real-time procedure as the medical instrument is inserted into the target point. In this way, the measuring application system 220 may display a video feed of the medical instrument being inserted into the phantom object 100 with real-time feedback to assist in the training of medical instrument insertion. Alternatively, as the user becomes more adept, the user may select an option in the application to not view the procedure and video feed in real time to more closely simulate a real-life procedure in which the user may not be able to view the inside of the organ (represented by the phantom object 100) of a live patient. As further described herein, the process 300 may be used to evaluate the performance of a medical device (e.g., virtual navigation system that renders a virtual insertion path within a headset). More specifically, the process 300 may be used to determine the accuracy of the virtual insertion path, and whether the virtual insertion path is accurately rendered for guiding the insertion of the medical instrument to the target location.

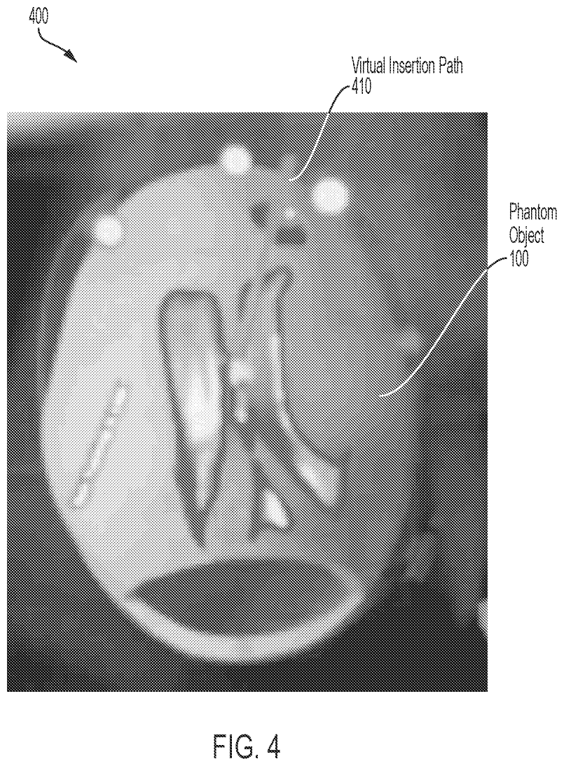

FIG. 4 illustrates an example virtual navigation system which may be evaluated using the techniques described herein in accordance with aspects of the present disclosure. As shown in FIG. 4, an example virtual navigation system may render a virtual insertion path 410 within the display of an AR headset through an interface 400. In the example shown in FIG. 4, the virtual navigation system renders the virtual insertion path 410 through a phantom object 100 representing a skull to aid a user in identifying the correct insertion path of a medical instrument during a medical procedure. The measurement system, described herein, may be used to verify the accuracy of this virtual insertion path 410. For example, the medical instrument may be inserted along the virtual insertion path 410, and using the techniques described herein, the measuring application system 220 may measure a distance between the medical instrument and the target. Based on the measurement, any necessary adjustments may be made to the virtual navigation system to correct any errors in the rendering of the virtual insertion path 410.

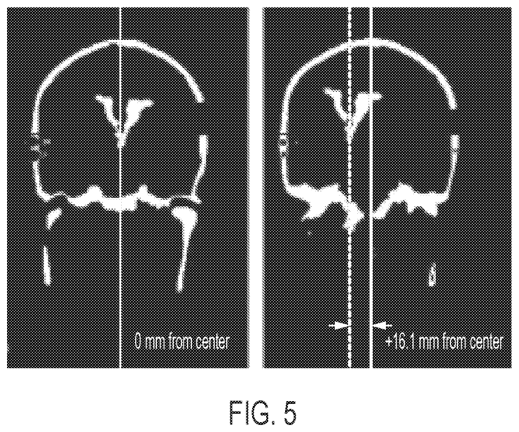

In some embodiments, the model of the skull, the positions of the fiducials, and the positions of the target spheres can be obtained from a CT scan of the phantom. Using data from another CT scan with a ventricle phantom, synthetic CT scans can be created, as shown in FIG. 5. Specifically, the spherical targets can be digitally removed from the CT scan and then an anatomical model, such as a ventricle model, can be placed such that its target feature (e.g., Foramen of Monro) is coincident with a specified target. These synthetic CT scans can be used in conjunction with the described phantom for training or validation activities. For example, the synthetic CT scan can be used when training for the conventional free-hand ventriculostomy procedure, where the surgeon would normally consult the CT scan.

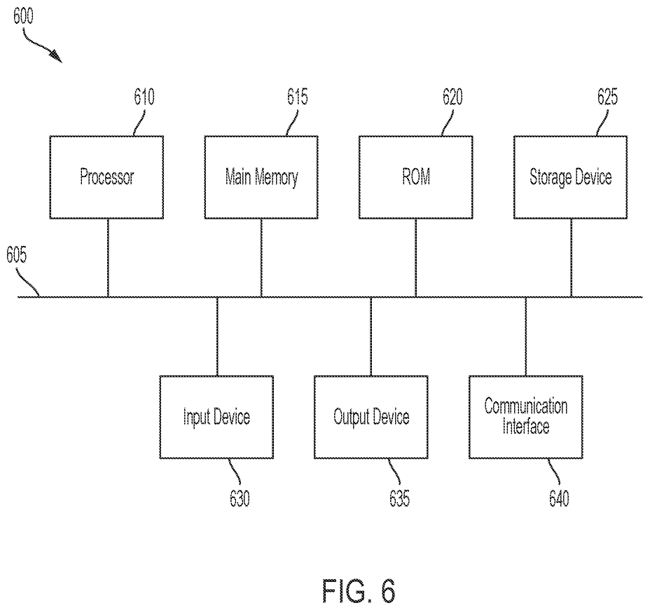

FIG. 6 illustrates example components of a device 600 that may be used within environment 200 of FIG. 2 or the system of FIG. 6. Device 600 may correspond to the phantom object 100 and/or the measuring application system 220. Each of the phantom object 100 and/or the measuring application system 220 may include one or more devices 600 and/or one or more components of device 600.

As shown in FIG. 6, device 600 may include a bus 605, a processor 610, a main memory 615, a read only memory (ROM) 620, a storage device 625, an input device 630, an output device 635, and a communication interface 640.

Bus 605 may include a path that permits communication among the components of device 600. Processor 610 may include a processor, a microprocessor, an application specific integrated circuit (ASIC), a field programmable gate array (FPGA), or another type of processor that interprets and executes instructions. Main memory 615 may include a random access memory (RAM) or another type of dynamic storage device that stores information or instructions for execution by processor 610. ROM 620 may include a ROM device or another type of static storage device that stores static information or instructions for use by processor 610. Storage device 625 may include a magnetic storage medium, such as a hard disk drive, or a removable memory, such as a flash memory.

Input device 630 may include a component that permits an operator to input information to device 600, such as a control button, a keyboard, a keypad, or another type of input device. Output device 635 may include a component that outputs information to the operator, such as a light emitting diode (LED), a display, or another type of output device. Communication interface 640 may include any transceiver-like component that enables device 600 to communicate with other devices or networks. In some implementations, communication interface 640 may include a wireless interface, a wired interface, or a combination of a wireless interface and a wired interface. In embodiments, communication interface 640 may receive computer readable program instructions from a network and may forward the computer readable program instructions for storage in a computer readable storage medium (e.g., storage device 625).

Device 600 may perform certain operations, as described in detail below. Device 600 may perform these operations in response to processor 610 executing software instructions contained in a computer-readable medium, such as main memory 615. A computer-readable medium may be defined as a non-transitory memory device and is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. A memory device may include memory space within a single physical storage device or memory space spread across multiple physical storage devices.

The software instructions may be read into main memory 615 from another computer-readable medium, such as storage device 625, or from another device via communication interface 640. The software instructions contained in main memory 615 may direct processor 610 to perform processes that will be described in greater detail herein. Alternatively, hardwired circuitry may be used in place of or in combination with software instructions to implement processes described herein. Thus, implementations described herein are not limited to any specific combination of hardware circuitry and software.

In some implementations, device 600 may include additional components, fewer components, different components, or differently arranged components than are shown in FIG. 6.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the disclosure may include a system, a method, and/or a computer program product at any possible technical detail level of integration. The computer program product may include a computer readable storage medium (or media) having computer readable program instructions thereon for causing a processor to carry out or execute aspects and/or processes of the present disclosure.

In embodiments, the computer readable program instructions may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on a user's computer, partly on the user's computer, as a stand-alone software package, partly on the user's computer and partly on a remote computer or entirely on the remote computer or server.

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

In embodiments, a service provider could offer to perform the processes described herein. In this case, the service provider can create, maintain, deploy, support, etc., the computer infrastructure that performs the process steps of the disclosure for one or more customers. These customers may be, for example, any business that uses technology. In return, the service provider can receive payment from the customer(s) under a subscription and/or fee agreement and/or the service provider can receive payment from the sale of advertising content to one or more third parties.

The foregoing description provides illustration and description, but is not intended to be exhaustive or to limit the possible implementations to the precise form disclosed. Modifications and variations are possible in light of the above disclosure or may be acquired from practice of the implementations.

It will be apparent that different examples of the description provided above may be implemented in many different forms of software, firmware, and hardware in the implementations illustrated in the figures. The actual software code or specialized control hardware used to implement these examples is not limiting of the implementations. Thus, the operation and behavior of these examples were described without reference to the specific software code—it being understood that software and control hardware can be designed to implement these examples based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While the present disclosure has been disclosed with respect to a limited number of embodiments, those skilled in the art, having the benefit of this disclosure, will appreciate numerous modifications and variations there from. It is intended that the appended claims cover such modifications and variations as fall within the true spirit and scope of the disclosure.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

What is claimed is:

1. A computer-implemented method comprising:
detecting a medical instrument within a phantom object based on sensor data captured by one or more sensors implemented within the phantom object;
measuring a distance between the medical instrument and a target point based on the sensor data; and
storing or outputting information identifying the distance between the medical instrument and the target point.

2. The computer-implemented method of claim 1, wherein the sensors comprise one or more cameras that capture video or image data.

3. The computer-implemented method of claim 2, wherein the detecting of the medical instrument is based on image or pixel-based classification of the video or image data.

4. The computer-implemented method of claim 1, further comprises detecting the target point, wherein the measuring the distance between the medical instrument and the target point is based on the detecting the target point.

5. The computer-implemented method of claim 4, wherein the detecting the target points comprises at least one selected from the group consisting of:
detecting one or more markers implemented within the phantom object from the sensor data; and
receiving user input identifying the target location from the sensor data.

6. The computer-implemented method of claim 1, wherein the phantom object is a replica of an organ and the medical instrument is a catheter or needle or replica of a catheter or needle.

7. The computer-implemented method of claim 1, wherein a synthetic medical image is generated based on some combination of the phantom object geometry, relevant anatomical features, and the identified target location.

8. The computer-implemented method of claim 1, wherein the distance between the medical instrument and the target point is measured during a training procedure or a procedure to evaluate performance of a system used to guide insertion of the medical instrument.

9. A computer program product comprising a computer readable storage medium having program instructions embodied therewith, the program instructions executable by a computing device to cause the computing device to perform operations comprising:
detecting a medical instrument within a phantom object based on sensor data captured by one or more sensors implemented within the phantom object;
measuring a distance between the medical instrument and a target point based on the sensor data; and
storing or outputting information identifying the distance between the medical instrument and the target point.

10. The computer program product of claim 9, wherein the sensors comprise one or more cameras that capture video or image data.

11. The computer program product of claim 10, wherein operations for detecting the medical instrument is based on image or pixel-based classification of the video or image data.

12. The computer program product of claim 9, wherein the operations further comprise detecting the target point, wherein the measuring the distance between the medical instrument and the target point is based on the detecting the target point.

13. The computer program product of claim 12, wherein operations for detecting the target points comprises at least one selected from the group consisting of:
detecting one or more markers implemented within the phantom object from the sensor data; and
receiving user input identifying the target location from the sensor data.

14. The computer program product of claim 9, wherein the phantom object is a replica of an organ.

15. The computer program product of claim 9, wherein a synthetic medical image is generated based on some combination of the phantom object geometry, relevant anatomical features, and the identified target location.

16. The computer program product of claim 9, wherein the distance between the medical instrument and the target point is measured during a training procedure or a procedure to evaluate performance of a system.

17. A system comprising:
a phantom object;
one or more sensors within the phantom object; and
a processor, a computer readable memory, a non-transitory computer readable storage medium associated with a computing device in communication with the phantom object, and program instructions executable by the computing device to cause the computing device to perform operations comprising:
detecting a medical instrument within the phantom object based on sensor data captured by the one or more sensors;
measuring a distance between the medical instrument and a target point based on the sensor data; and
storing or outputting information identifying the distance between the medical instrument and the target point.

18. The system of claim 17, wherein the phantom object is a replica of an organ and the medical instrument is a catheter or needle or replica of a catheter or needle.

19. The system of claim 17, wherein the phantom object comprises one or more markers used to define the target point.

20. The system of claim 17, wherein the phantom object includes an inner compartment that can be filled with tissue-mimicking material.

21. The system of claim 17, wherein the one or more sensors comprise one or more cameras, the phantom object comprising one or more internal lights to support sensing via the one or more cameras.

22. The system of claim 17, wherein the detecting the medical instrument is based on image or pixel-based classification.

23. The system of claim 17, wherein a synthetic medical image is generated based on some combination of the phantom object geometry, relevant anatomical features, and the identified target location.
